(19) United States
(12) Patent Application Publication
(10) Pub. No.: US 2016/0225291 A1
(43) Pub. Date: Aug. 4, 2016

(54) SYSTEM AND METHOD FOR DISPLAYING MEDICAL IMAGES

(71) Applicant: Infinitt Healthcare Co., Ltd., Seoul (KR)

(72) Inventors: Dong Hoon Yu, Seoul (KR); Han Young Kim, Seoul (KR); Yu Hyeon Choi, Incheon (KR); Dae Gyu Min, Incheon (KR); Tae Jun Choi, Seoul (KR)

(73) Assignee: Infinitt Healthcare Co., Ltd.

(21) Appl. No.: 15/008,880

(22) Filed: Jan. 28, 2016

(30) Foreign Application Priority Data
Jan. 29, 2015 (KR) 10-2015-0014008

(51) Int. Cl. G09B 23/30; G06T 19/00; G06T 17/00

(52) U.S. Cl. CPC G09B 23/303; G06T 17/00; G06T 19/006; G06T 2210/41; A61B 2034/104

(57) ABSTRACT

A medical image display system and method are disclosed herein. The medical image display system includes a processor within a computing system. The processor acquires an image of the body part of a subject where a stent is to be implanted and a three-dimensional (3D) anatomical model which is generated to include the body part, simulates variation in the 3D anatomical model, attributable to the implantation of the stent, for each of a plurality of simulation conditions, including one or more of a type of stent and a target location where the stent is to be implanted, based on the 3D anatomical model, and displays simulation results for the plurality of respective simulation conditions on a screen by using variations in the 3D anatomical model.

### CROSS-REFERENCE TO RELATED APPLICATION

This application claims under 35 U.S.C. §119(a) the benefit of Korean Patent Application No. 10-2015-0014008 filed on Jan. 29, 2015, which is incorporated herein by reference.

### TECHNICAL FIELD

The present invention relates generally to a system and method for displaying medical images, and more particularly to a system and method that can more efficiently display a simulation image regarding a stent that is to be implanted in the body of a subject.

### BACKGROUND ART

Recently, due to reasons such as western dietary habits, the extension of the average life span, lack of exercise, etc., coronary artery diseases have rapidly increased. Coronary artery diseases occur because a coronary artery is narrowed or occluded due to stenosis and thus the metabolic demand of the heart muscle is not satisfied. A representative treatment method for coronary artery diseases is stent implantation, which is performed to mitigate stenosis by inserting a metallic mesh tube, i.e., a stent, into a lesion and then inflating it.

Stent implantation is a nonsurgical treatment method, and is advantageous in that physical, psychological and economic burdens from which a patient suffers are small because minimum incision, anesthesia and invasive manipulation are employed. However, stent implantation is disadvantageous in that it is difficult to ensure the accuracy of a medical procedure with a high level of difficulty because the medical procedure is dependent upon two-dimensional X-ray angiogram (2D XA) images and thus the understanding of a three-dimensional (3D) structure is dependent upon the intuition of a medical team and tactile feedback.

Furthermore, with the advancement of stents, stent implantation considerably reduces serious complications, such as the acute occlusion of a blood vessel, which may be caused by conventional coronary balloon angioplasty, to less than 1%, and enables coronary intervention to be applied to complicated lesions, such as a multi-vessel disease, an opening lesion, a bifurcation lesion, a left main artery lesion, etc. However, stent implantation still has the important problem of in-stent re-stenosis.

It is known that in-stent stenosis is related to extracellular matrix formation attributable to the rapid propagation of smooth muscle cells having moved from the media of a blood vessel, i.e., neointimal hyperplasia, platelet thrombus, or inflammatory reaction. It is reported that in-stent re-stenosis exhibited a disease rate ranging from about 15 to 20% for a simple lesion and a disease rate ranging from about 30 to 60% for a complicated lesion, such as a lesion of a diabetes patient, a bifurcation lesion, or the like.

As the number of stent implantation cases increases, the absolute frequency of in-stent re-stenosis also increases. Furthermore, the number of recurrences of re-stenosis after the percutaneous treatment of in-stent re-stenosis gradually increases. These problems have become new problems in the field of coronary intervention.

Conventionally, although many stent manufacture-related developments, such as a change in the structure of a stent and a change in the material of a stent, have been attempted in order to overcome the above problems, there may be a situation in which the life of a patient is threatened due to the misjudgment of a doctor because which of the numerous types of existing stents is most appropriate for the implantation target portion of the corresponding patient must be determined totally based on the knowledge and experience of the doctor. That is, the existing well-known technologies do not provide appropriate criteria or an appropriate method for the determination of which stent is most effective from a physiological or physical perspective.

Meanwhile, U.S. Patent Application No. 2013-0144573 entitled “Method and System for Patient-Specific Hemodynamic Assessment of Virtual Stent Implantation” discloses a method for assessment of virtual stent implantation in an aortic aneurysm, comprising: generating a patient-specific 4D anatomical model of the aorta from the 4D medical imaging data; adjusting a model representing mechanical properties of the aorta wall to reflect changes due to aneurysm growth at a plurality of time stages; generating a stable deformation configuration of the aorta for each of the plurality of time stages by performing fluid structure interaction (FSI) simulations using the patient-specific 4D anatomical model at each time stage based on the adjusted model representing the mechanical properties of the aorta wall at each time stage; performing virtual stent implantation for each stable deformation configuration of the aorta; and performing FSI simulations for each virtual stent implantation.

That is, this preceding technology is used to model the aorta of a specific patient using medical images and then assess virtual stent implantation based on the modeled aorta in a thermodynamic manner, and has the advantage of checking the effect of implantation by virtually simulating stent implantation before actual stent implantation. However, this preceding technology is disadvantageous in that a user cannot easily check the effect of simulation because there is provided a display structure in which a user cannot easily check the result values of the simulation at one time.

### SUMMARY OF THE DISCLOSURE

At least one embodiment of the present invention is intended to more efficiently display a simulation image regarding a stent that is to be implanted in the body of a subject.

At least one embodiment of the present invention is intended to display simulation results for a plurality of respective simulation conditions on a screen in conjunction with each other so that variations in a 3D anatomical model can be contrasted with each other.

At least one embodiment of the present invention is intended to simultaneously display simulation results for a plurality of types of stents on a single screen, thereby providing the simulation results in a form in which it is easy for a user to more intuitively understand the simulation results.

At least one embodiment of the present invention is intended to display the simulation results of the implantation of a stent, which has been virtually performed, in a form that is easy to view.

At least one embodiment of the present invention is intended to aid in the selection of the most appropriate stent that is to be implanted in a subject.

According to an aspect of the present invention, there is provided a medical image display system, including a processor. The processor includes an image acquisition unit, an image acquisition unit, and a display control unit.

The image acquisition unit is configured to acquire an image of the body part of a subject where a stent is to be implanted and a three-dimensional (3D) anatomical model which is generated to include the body part; a simulation unit is configured to simulate variation in the 3D anatomical model, attributable to the implantation of the stent, for each of a plurality of simulation conditions, including one or more of a type of stent and a target location where the stent is to be implanted, based on the 3D anatomical model; and a display control unit is configured to display simulation results for the plurality of respective simulation conditions on a screen by using variations in the 3D anatomical model.

The display control unit may be further configured to display the simulation results for the plurality of respective simulation conditions on the screen in conjunction with each other so that the variations in the 3D anatomical model can be contrasted with each other; may be further configured to display the simulation results for the plurality of respective simulation conditions on the screen based on the same reference point within the 3D anatomical model; may be further configured to display the simulation results for the plurality of respective simulation conditions on the screen based on a reference point or a reference direction at or in which the variations in the 3D anatomical model are maximized, and may be further configured to display one or more evaluation criteria, selected according to the predetermined setting or input of a user, on the 3D anatomical model based on the simulation results for the plurality of respective simulation conditions.

The medical image display system may further include a first interface control unit configured to provide a user menu that enables a user to select one or more evaluation criteria to be displayed on the screen for the simulation results; and the display control unit may be further configured to display the simulation results on the screen based on the evaluation criteria selected according to the response of the user via the user menu.

The medical image display system may further include a second interface control unit configured to provide a user menu that enables a user to select a reference location at which numerical values of one or more evaluation criteria are to be displayed, and a first identification unit configured to identify the reference location from the input of the user via the user menu; and the display control unit may be further configured to display the numerical values of the evaluation criteria on the 3D anatomical model, displayed as the simulation results for each of the plurality of simulation conditions, at the reference location.

The display control unit may be further configured to display the evaluation criteria, including one or more of a fractional flow reserve (FFR) value, a wall stress shear (WSS) value, a velocity value and a wall stress value, on the 3D anatomical model. The medical image display system may further include: a second identification unit configured to identify the input of a user corresponding to any one of a plurality of images displayed on the display screen as the simulation results for the plurality of respective simulation conditions; and a database configured to store information, including the image corresponding to the input of the user, in response to the identified input of the user.

According to another aspect of the present invention, there is provided a medical image display method, including: acquiring an image of the body part of a subject where a stent is to be implanted and a three-dimensional (3D) anatomical model which is generated to include the body part; simulating variation in the 3D anatomical model, attributable to implantation of the stent, for each of a plurality of simulation conditions, including one or more of a type of stent and a target location where the stent is to be implanted, based on the 3D anatomical model; and displaying simulation results for the plurality of respective simulation conditions on a screen by using variations in the 3D anatomical model.

The displaying may include displaying the simulation results for the plurality of respective simulation conditions on the screen in conjunction with each other so that the variations in the 3D anatomical model can be contrasted with each other; may include displaying the simulation results for the plurality of respective simulation conditions on the screen based on the same reference point within the 3D anatomical model; may include displaying the simulation results for the plurality of respective simulation conditions on the screen based on a reference point or a reference direction at or in which the variations in the 3D anatomical model are maximized; and may include displaying one or more evaluation criteria, selected according to the predetermined setting or input of a user, on the 3D anatomical model based on the simulation results for the plurality of respective simulation conditions.

The medical image display method may further include providing a user menu that enables a user to select one or more evaluation criteria to be displayed on the screen for the simulation results; and the displaying may include displaying the simulation results on the screen based on the evaluation criteria selected according to the response of the user via the user menu.

The medical image display method may further include providing a user menu that enables a user to select a reference location at which the numerical values of one or more evaluation criteria are to be displayed, and identifying the reference location from the input of the user via the user menu; and the displaying may include displaying the numerical values of the evaluation criteria on the 3D anatomical model, displayed as the simulation results for each of the plurality of simulation conditions, at the reference location.

The displaying may include displaying the evaluation criteria, including one or more of a fractional flow reserve (FFR) value, a wall stress shear (WSS) value, a velocity value and a wall stress value, on the 3D anatomical model. The medical image display method may further include: identifying the input of a user corresponding to any one of a plurality of images displayed on the display screen as the simulation results for the plurality of respective simulation conditions; and storing information, including the image corresponding to the input of the user, in a database in response to the identified input of the user.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a diagram showing the schematic configuration of a medical image display system according to an embodiment of the present invention;

FIG. 2 is a first block diagram of a medical image display system according to an embodiment of the present invention;

FIG. 3 is a second block diagram of a medical image display system according to an embodiment of the present invention;

FIG. 4 is a third block diagram of a medical image display system according to an embodiment of the present invention;

FIG. 5 is a diagram showing a first embodiment in which medical images are displayed on a screen according to an embodiment of the present invention;

FIG. 6 is a diagram showing a second embodiment in which medical images are displayed on a screen according to an embodiment of the present invention;

FIG. 7 is a diagram showing a third embodiment in which medical images are displayed on a screen according to an embodiment of the present invention;

FIGS. 8A and 8B are views showing a fourth embodiment in which medical images are displayed on a screen according to an embodiment of the present invention;

FIGS. 9A and 9B are views showing a fifth embodiment in which medical images are displayed on a screen according to an embodiment of the present invention; and

FIG. 10 is an operation flowchart of a medical image display method according to an embodiment of the present invention.

### DETAILED DESCRIPTION OF THE DISCLOSURE

Embodiments of the present invention will be described in detail below with reference to the accompanying drawings. In the following description, detailed descriptions of related known elements or functions that may unnecessarily make the gist of the present invention obscure will be omitted. Furthermore, in the following description of embodiments of the present invention, specific numerical values are merely examples.

The present invention relates to a medical image display system and method, and more particularly to system and method that can more efficiently display a simulation image regarding a stent that is to be implanted in the body of a subject.

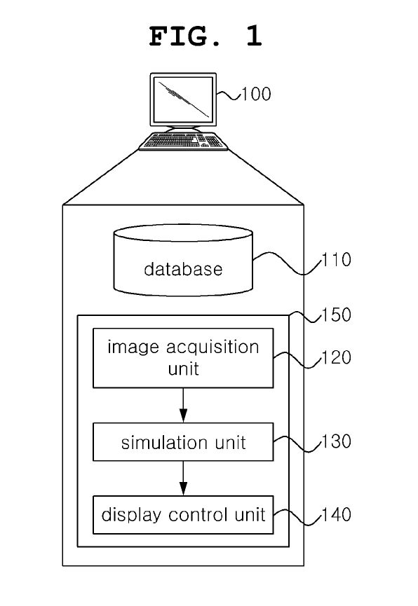

FIG. 1 is a diagram showing the schematic configuration of a medical image display system 100 according to an embodiment of the present invention.

Referring to FIG. 1, the medical image display system 100 according to the present embodiment includes an image acquisition unit 120, a simulation unit 130, and a display control unit 140. Alternatively, the medical image display system 100 may include a database 110 and a processor 150, and the processor 150 may include the image acquisition unit 120, the simulation unit 130 and the display control unit 140.

The image acquisition unit 120 acquires an image of the body part of the subject where a stent is to be implanted, and a 3D anatomical model which is generated to include the body part.

In this case, the image of the body part of the subject acquired by the image acquisition unit 120 may be an image of the body part, before the implantation of the stent, which is acquired to virtually simulate the implantation of the stent before a doctor actually implants the stent in the body of the subject.

Furthermore, the image acquisition unit 120 may acquire a 3D anatomical model generated to include the body part of the subject, particularly a 3D anatomical model including the structure of blood vessels.

In this case, the present invention may further include a modeling unit (not shown) configured to acquire the 3D anatomical model. The modeling unit may generate a 3D anatomical model including the structure of blood vessels based on the image acquired by the image acquisition unit 120.

The medical image display method according to the present invention performs simulation based on the generated 3D anatomical model, thereby displaying a simulation result image on a screen.

The simulation unit 130 simulates variation in the 3D anatomical model, attributable to the implantation of the stent, for each of a plurality of simulation conditions (which may be construed as stent implantation conditions) including one or more of the type of stent and a target location where the stent is to be implanted based on the 3D anatomical model generated based on the image acquired by the image acquisition unit 120. In this case, the type of stent may include stent size, shape, material, strength and flexibility. Furthermore, an atmospheric pressure (ATM) condition in which the stent is inflated may be additionally taken into account.

In this case, a stent ATM is representative of the atmospheric pressure at which a stent is inflated, i.e., the extent to which a stent is inflated. Different types of stents may have different optimum stent ATM values. That is, the ATM at which damage to the wall of a blood vessel is minimized when a stent is inflated is dependent on the type of stent. In the present embodiment, the simulation unit 130 may perform simulation for each type of stent while taking into account conditions, such as stent size, shape, material, etc., in addition to the stent ATM condition, as described above.

The display control unit 140 may display simulation results for the plurality of respective simulation conditions on the screen by using variations in the 3D anatomical model. That is, the display control unit 140 displays the 3D anatomical model, varied according to the simulation results for the plurality of respective simulation conditions, on the screen.

Furthermore, the display control unit 140 may simultaneously display simulation images for the plurality of stent implantation conditions on a single screen.

Furthermore, the display control unit 140 may display simulation results for the plurality of respective simulation conditions (or stent implantation conditions) on the screen in conjunction with each other so that variations in the 3D anatomical model can be contrasted with each other.

In this case, the present invention may simultaneously display both an image before the implantation of a stent and a simulation image after the implantation of the stent on a single screen by means of the display control unit 140, and thus a user (or a doctor) may check and compare results before and results after the implantation of the stent at one time. Furthermore, the present invention may simultaneously display simulation result images, when a plurality of types of stents is implanted, for a single evaluation criterion on a single screen, and thus a user may check simulation results for the plurality of types of stents at one time.

Furthermore, the display control unit 140 may display the simulation results for the plurality of respective simulation conditions on the screen based on the same reference point within the 3D anatomical model. That is, the display control unit 140 may display a plurality of simulation result images for the plurality of respective simulation conditions on a single screen based on the same reference point within the 3D anatomical model. In this case, the display control unit 140 may display simulation result images based on both the same reference point within the 3D anatomical model and the same viewpoint. As described above, the display control unit 140 may use the same reference feature point in order to perform comparison between simulation results or between states before and after simulation.

Furthermore, the display control unit 140 may display the simulation results for the plurality of respective simulation conditions on the screen based on a reference point or a reference direction (or a viewpoint) at or in which the variations in the 3D anatomical model are maximized. That is, the display control unit 140 may display an image related to reference point or a viewpoint at which variation in wall stress or velocity is viewed best on a screen according to simulation results.

Furthermore, the display control unit 140 may display one or more evaluation criteria, selected according to the predetermined setting or input of the user (which may be newly input or modified), on the 3D anatomical model based on the simulation results for the plurality of respective simulation conditions. In other words, the display control unit 140 may previously set one or more evaluation criterion conditions that are simultaneously displayed on simulation result images output onto a screen, and may receive one or more evaluation criterion conditions from the user. In this case, the user is a person who desires to check simulation results, and may be a radiologist or a blood vessel-related physician.

Furthermore, the setting of the user refers to setting that is previously performed by a user. For example, the setting of the user may refer to the setting of the number of evaluation criteria items of a simulation result image to be displayed (i.e., the number of items related to the display of a result image for FFR or to the simultaneous display of result images for both FFR and WSS), or the setting of the number of stents (the setting of the display of a result image for a single stent, the setting of the display of result images for three stents, or the like). In this case, the evaluation criteria include fractional flow reserve (FFR), wall stress shear (WSS), velocity, wall stress, etc. In this case, FFR refers to the ratio between the maximum blood flow rates of distal and proximal normal blood vessels in a coronary artery stenosis portion.

The database 110 may store an image (in particular, a generated 3D anatomical model, an image before the implantation of a stent in the body of a subject, or the like) acquired by the image acquisition unit 120, and may also store simulation result data for each of a plurality of types of stents.

Furthermore, the database 110 may store information including an image corresponding to the input of the user in response to the input of the user. That is, if a user stores information about the appropriate one of a plurality of simulation result images displayed on a screen or selects the most appropriate image in order to report it to another person (for example, the appropriate image may be selected by double-clicking on the image, or the input of the user may be received by being equipped with a separate image selection menu), the image selected by the user or the image-related simulation information may be stored in the database 110.

The additional components of the system 100 of the present embodiment may be described in greater detail below with reference to the accompanying drawings. In this case, the system 100 of the present embodiment may further include a first or second interface control unit and a first or second identification unit, and the interface control units may refer to modules corresponding to given corresponding functions. Descriptions thereof are given below in greater detail.

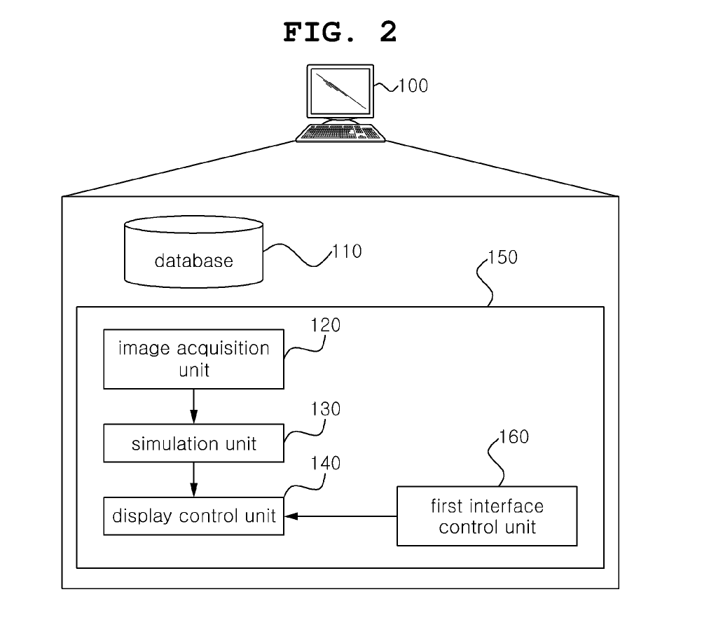

FIG. 2 is a first block diagram of a medical image display system according to an embodiment of the present invention.

Referring to FIG. 2, the system 100 of the present embodiment may further include a first interface control unit 160 in addition to the image acquisition unit 120, the simulation unit 130 and the display control unit 140 present in the processor 150 of FIG. 1.

The first interface control unit 160 provides a user menu that enables a user to select an evaluation criterion to be displayed on a screen for simulation results.

In this case, the first interface control unit 160 may provide FFR, WSS, velocity, wall stress, etc. as a user menu (in this case, the user menu may be viewed as an evaluation criterion selection menu) selectable by the user, as described above. Furthermore, the first interface control unit 160 may identify the input of the user input from the evaluation criterion selection menu.

Furthermore, the display control unit 140 may display the simulation results on the screen based on the evaluation criterion selected based on the response of the user via the user menu. If the user selects “pressure” in the evaluation criterion selection menu, the display control unit 140 may display simulation result images for three respective stents on a screen as an example. In this case, an image before the implantation of stents and simulation result images for the three respective stents (i.e., four images) are output together on a display screen. This enables the user to check simulation results for respective stents on a single screen. Furthermore, the display control unit 140 may control the screen arrangement of images to be output in response to the input of the user.

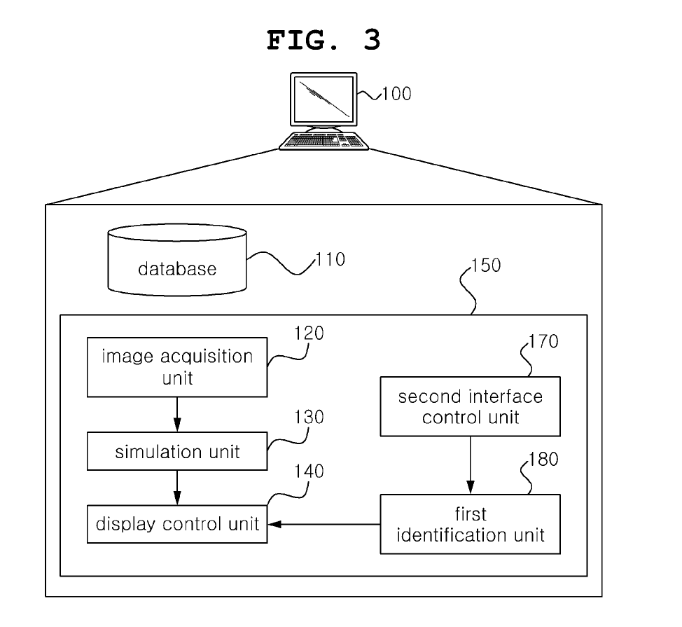

FIG. 3 is a second block diagram of a medical image display system according to an embodiment of the present invention.

Referring to FIG. 3, the system 100 of the present embodiment may further include a second interface control unit 170 and a first identification unit 180 in addition to the image acquisition unit 120, the simulation unit 130, and the display control unit 140 present in the processor 150 of FIG. 1.

The second interface control unit 170 provides a user menu that enables a user to select a reference location where the numerical value of at least one evaluation criterion is to be displayed. In this case, the user menu may display a state numerical value for the selected reference location in connection with each display image even when the reference location is selected in any one of images simultaneously displayed on a screen, including an image before the implantation of a stent. That is, the display control unit 140 may display a state numerical value for a specific location (i.e., a reference location), designated by the user, in connection with each of display images by synchronizing results for respective simulations.

The first identification unit 180 identifies the reference location from the input of the user (i.e., the input of the user adapted to select the reference location) via the user menu in order to display a simulation numerical value at a reference location received from the user.

Furthermore, the display control unit 140 displays the numerical value of the evaluation criterion on the 3D anatomical model, displayed as the simulation results for each of the plurality of simulation conditions, at the reference location. In this case, the display control unit 140 may display at least one of FFR, WSS, velocity and pressure values as an evaluation criterion numerical value that is displayed on the 3D anatomical model.

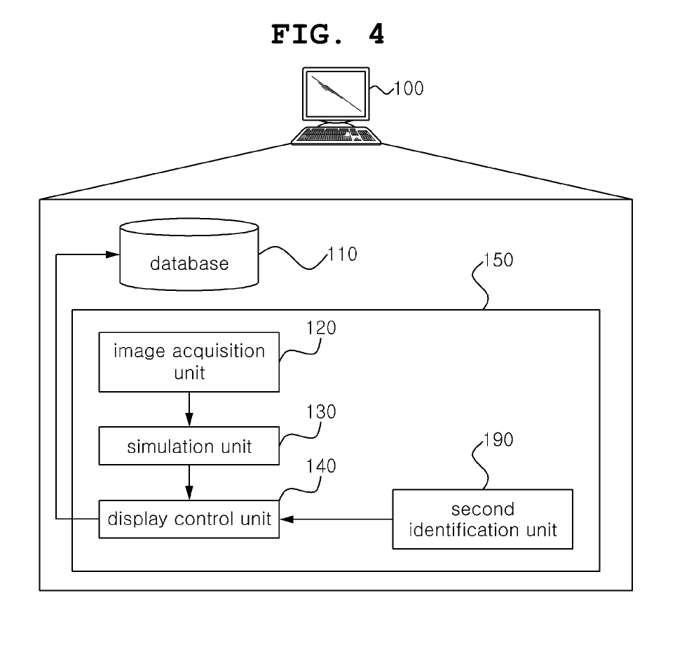

FIG. 4 is a third block diagram of a medical image display system according to an embodiment of the present invention.

Referring to FIG. 4, the system 100 of the present embodiment may further include a second identification unit 190 in addition to the image acquisition unit 120, the simulation unit 130, and the display control unit 140 present in the processor 150 of FIG. 1, and the system 100 may include a database 110 and the processor 150.

The second identification unit 190 identifies the input of the user corresponding to any one of a plurality of images displayed on the display screen as simulation results for the plurality of simulation conditions.

In this case, the input of the user identified by the second identification unit 190 may be input adapted to store information about the most appropriate image of a plurality of images displayed on the display screen as results or a report. For this purpose, the display control unit 140 may provide an image selection menu to a partial region of a displayed image.

Furthermore, the processor 150 stores information including an image corresponding to the input of the user in the database 110 in response to the input of the user identified by the second identification unit 190. That is, the database 110 may store the image selected by the input of the user (i.e., information about one of simulation result images, which is determined to be most appropriate by the user).

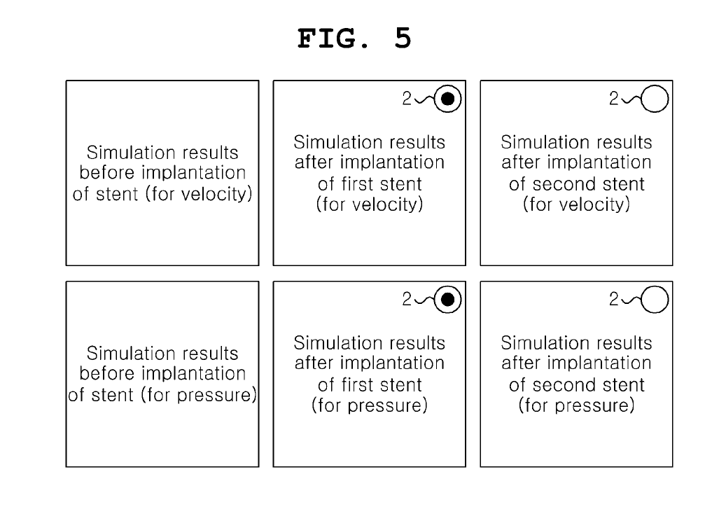

FIG. 5 is a diagram showing a first embodiment in which medical images are displayed on a screen according to an embodiment of the present invention.

Referring to FIG. 5, simulation images before the implantation of stents and simulation images after the implantation of the stents may be simultaneously displayed on a display screen according to the present invention.

Furthermore, the screen of FIG. 5 is a screen that is displayed when information about velocity and wall stress is received as evaluation criteria according to the input of a user or the setting of a user and information about two of a plurality of types of stents is received. That is, when two evaluation criteria and two stents have been selected, simulation result images according to the present embodiment may be displayed, as shown in FIG. 5.

Furthermore, an image selection menu 2 may be displayed on a partial region of a simulation result image, and the user may click on the image selection menu 2 of an image to be stored in order to store the most appropriate image of simulation result images as results or a report, thereby storing the clicked corresponding image in the database.

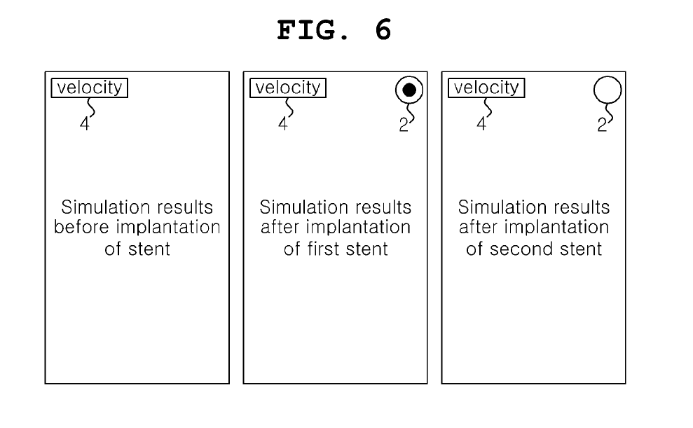

FIG. 6 is a diagram showing a second embodiment in which medical images are displayed on a screen according to an embodiment of the present invention.

Referring to FIG. 6, FIG. 6 shows only simulation result images regarding the velocity of evaluation criteria, and shows an example in which result images of the simulations of the implantation of stents 1 and 2, together with an image before the implantation of the stents, are displayed on a single screen.

Furthermore, an evaluation criterion selection menu 4 that enables an evaluation criterion, to be displayed on a screen, to be selected may be provided to a part of each of the displayed simulation result images, and also an image selection menu 2 that enables the most appropriate one of the simulation result images to be selected may be provided to another part of each corresponding simulation result image. In this case, the input of the user via the evaluation criterion selection menu 4 may be identified by the first interface control unit 160 of FIG. 2, and the input of the user via the image selection menu 2 may be identified by the second identification unit 190 of FIG. 4. Since descriptions thereof have been given in detail above, they are omitted.

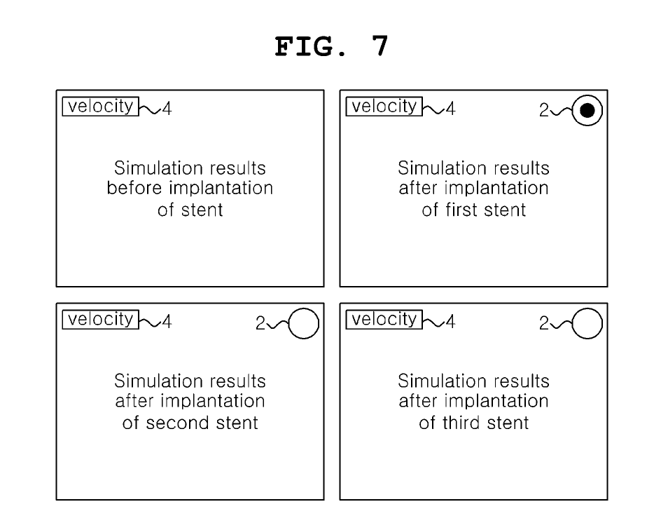

FIG. 7 is a diagram showing a third embodiment in which medical images are displayed on a screen according to an embodiment of the present invention.

Referring to FIG. 7, FIG. 7 shows only simulation result images regarding the velocity of evaluation criteria, and shows an example in which result images of the simulations of the implantation of stents 1, 2 and 3, together with an image before the implantation of the stents, are displayed on a single screen. That is, the medical image screen according to the present invention may be displayed according to the selection of the number of stents, as shown in FIG. 6 (in the case where the number of stents is 2), or FIG. 7 (in the case where the number of stents is 3).

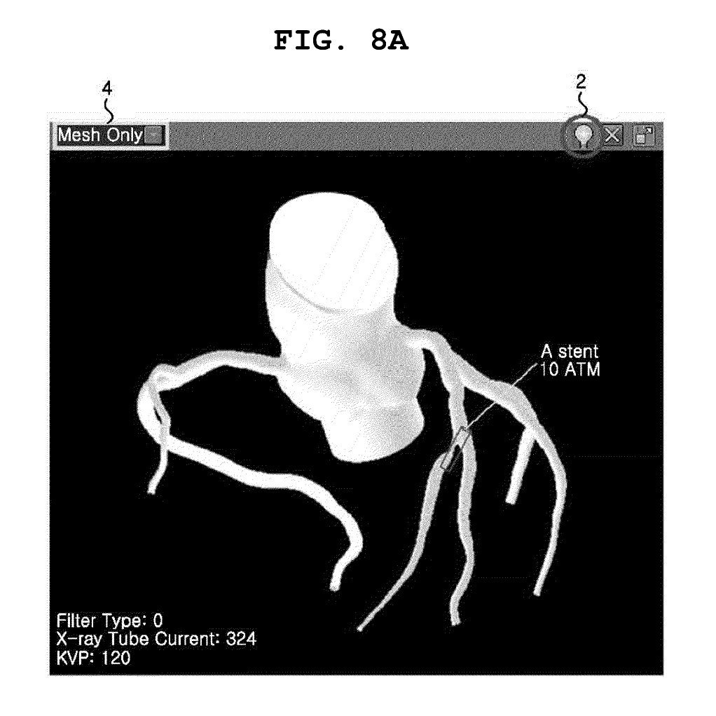

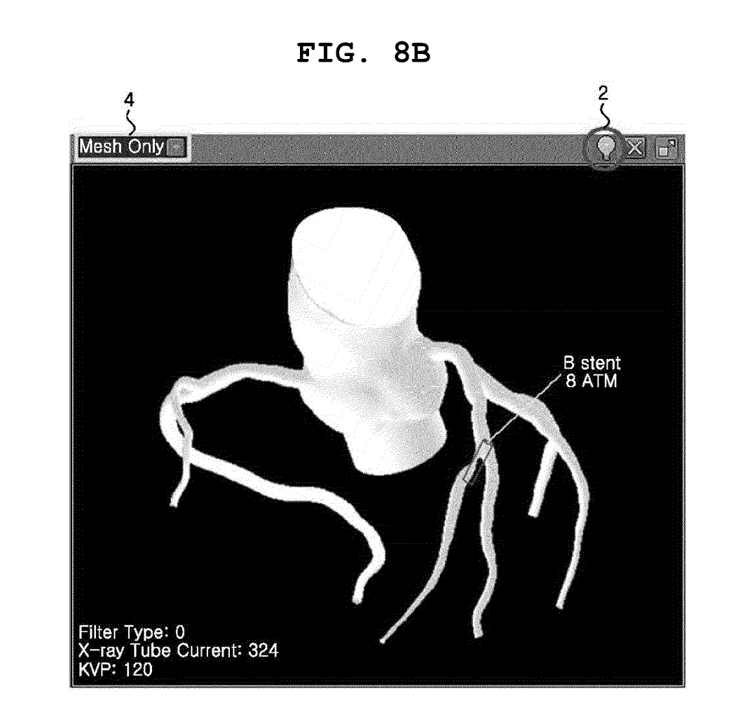

FIGS. 8A and 8B are views showing a fourth embodiment in which medical images are displayed on a screen according to an embodiment of the present invention.

Referring to FIGS. 8A and 8B, FIG. 8A shows a simulation image of stent A, and FIG. 8B shows a simulation image of stent B. In this case, 10 ATM and 8 ATM have been applied to stent A and stent B, respectively, and an evaluation criterion has been selected as “Mesh Only.” Furthermore, it may be seen that an evaluation criterion selection menu 4 is provided at the upper left end of each of the simulation result images and an image selection menu 2 is provided at the upper right end thereof.

Furthermore, when the user desires to store the image of FIG. 8A in the database as results or a report, the image selection menu 2 may be displayed in different colors (for example, the image selection menu that has not been selected may be displayed in gray, and the image selection menu that has been finally selected by the user may be displayed in yellow) or in different shapes by clicking on the image selection menu 2 of FIG. 8A.

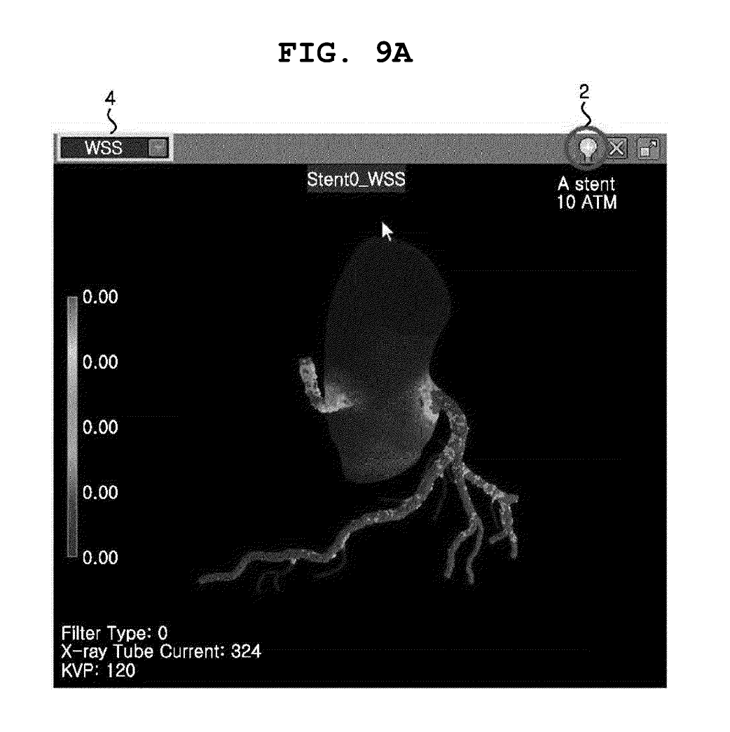

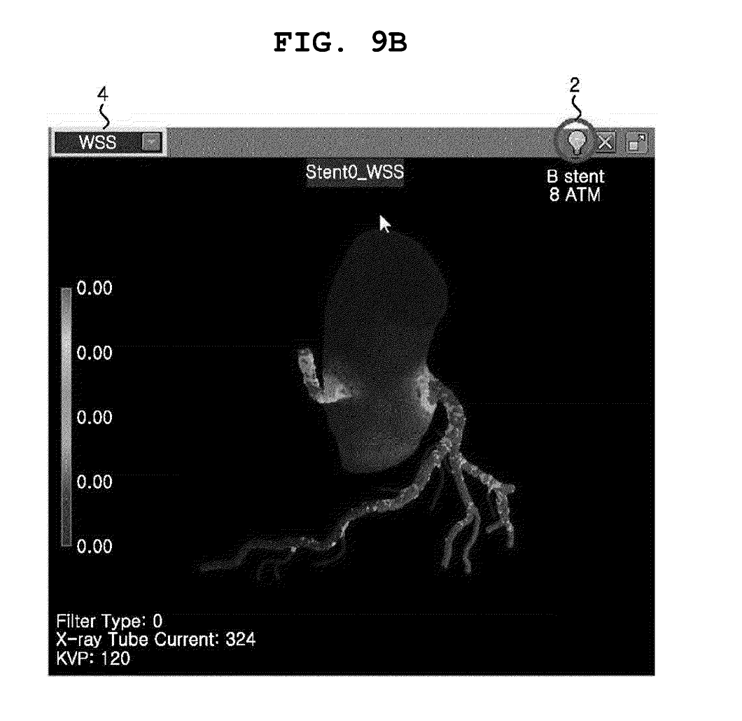

FIGS. 9A and 9B are views showing a fifth embodiment in which medical images are displayed on a screen according to an embodiment of the present invention.

Referring to FIGS. 9A and 9B, FIGS. 9A and 9B show simulation result images of two stents A and B regarding WSS of evaluation criteria. In the same manner as in FIGS. 8A and 8B, from FIGS. 9A and 9B, it can be seen that 10 ATM and 8 ATM have been applied to stent A and stent B, respectively, and the images of FIGS. 9A and 9B has been selected as a result or report image by a user.

Furthermore, according to the present invention, stents may be implanted and simulated at the location of a reference point on the 3D anatomical model of FIGS. 9A and 9B. The two images of FIGS. 9A and 9B show result images regarding stents that are implanted at the location of the reference point. In the simulation result images, WSS is in a desirable state when a blood vessel is displayed in light gray and in an undesirable state when a blood vessel is displayed in dark gray. It can be seen that stent A is more appropriate for a subject because the lower-left portion of a blood vessel where stent A has been implanted is displayed in light gray in FIG. 9A and the lower-left portion of a blood vessel where stent B has been implanted is displayed in dark gray in FIG. 9B.

Based on the detailed description given above, an operation flowchart of the present invention is described in brief.

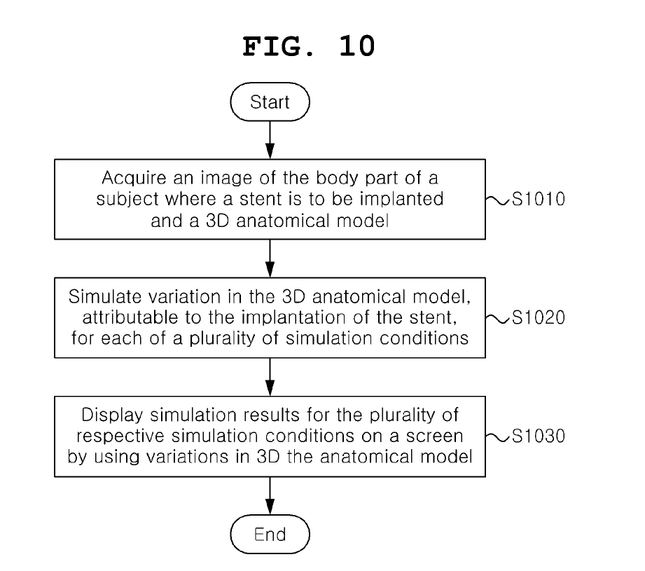

FIG. 10 is an operation flowchart of a medical image display method according to an embodiment of the present invention.

Referring to FIG. 10, first, the medical image display method according to the present embodiment includes step S1010 of acquiring, by the image acquisition unit 120 of the medical image display system 100, an image of the body part of a subject where a stent is to be implanted and a 3D anatomical model which is generated to include the body part.

In this case, at step S1010, the image acquisition unit 120 may acquire an image of the body part, before the real implantation of the stent, which is acquired to virtually simulate the implantation of the stent before a doctor actually implants the stent in the body of the subject, and may acquire a 3D anatomical model generated to include the body part of the subject, particularly a 3D anatomical model including the structure of blood vessels.

For this purpose, at step S1010, a 3D anatomical model including the structure of blood vessels may be generated based on the image acquired by the image acquisition unit 120.

Thereafter, at step S1020, the simulation unit 130 simulates variation in the 3D anatomical model, attributable to the implantation of the stent, for each of a plurality of simulation conditions (which may be construed as stent implantation conditions) including one or more of the type of stent and a target location where the stent is to be implanted based on the 3D anatomical model generated based on the image acquired at step S1010.

In this case, at step S1020, the simulation unit 130 may simulate variation in the 3D anatomical model while taking into account stent size, shape, material, strength, flexibility, and an ATM condition in which the stent is inflated as the types of stent. Since the description thereof has been given in detail above, it is omitted.

Thereafter, the display control unit 140 may display simulation results for the plurality of respective simulation conditions on a screen using the variation in the 3D anatomical model at step S1030.

That is, at step S1030, the display control unit 140 may display the 3D anatomical model, varied according to the simulation results for the plurality of respective simulation conditions, on the screen.

Furthermore, at step S1030, the display control unit 140 may display simulation results for the plurality of respective simulation conditions on the screen in conjunction with each other so that variations in the 3D anatomical model can be contrasted with each other; may display the simulation results for the plurality of respective simulation conditions on the screen based on the same reference point within the 3D anatomical model; may display the simulation results for the plurality of respective simulation conditions on the screen based on a reference point or a reference direction at or in which the variations in the 3D anatomical model are maximized; and may display one or more evaluation criteria, selected according to the setting or input of the user, on the 3D anatomical model based on the simulation results for the plurality of respective simulation conditions. Since the descriptions thereof have been given in detail above and the embodiments thereof have been described in detail above, they are omitted.

The medical image display method according to the embodiment of the present invention may be implemented in the form of program instructions that can be executed by a variety of computer means, and may be stored in a computer-readable storage medium. The computer-readable storage medium may include program instructions, a data file, and a data structure solely or in combination. The program instructions that are stored in the medium may be designed and constructed particularly for the present invention, or may be known and available to those skilled in the field of computer software. Examples of the computer-readable storage medium include magnetic media such as a hard disk, a floppy disk and a magnetic tape, optical media such as CD-ROM and a DVD, magneto-optical media such as a floptical disk, and hardware devices particularly configured to store and execute program instructions such as ROM, RAM, and flash memory. Examples of the program instructions include not only machine language code that is constructed by a compiler but also high-level language code that can be executed by a computer using an interpreter or the like. The above-described hardware components may be configured to act as one or more software modules that perform the operation of the present invention, and vice versa.

At least one embodiment of the present invention has the advantage of more efficiently displaying a simulation image regarding a stent that is to be implanted in the body of a subject.

At least one embodiment of the present invention has the advantage of displaying simulation results for a plurality of respective simulation conditions on a screen in conjunction with each other so that variations in a 3D anatomical model can be contrasted with each other.

At least one embodiment of the present invention has the advantage of simultaneously displaying simulation results for a plurality of types of stents on a single screen, thereby providing the simulation results in a form in which it is easy for a user to more intuitively understand the simulation results.

At least one embodiment of the present invention has the advantage of displaying the simulation results of the implantation of a stent, which has been virtually performed, in a form that is easy to view.

At least one embodiment of the present invention has the advantage of acquiring an image of the body part of a subject where a stent is to be implanted and a 3D anatomical model which is generated to include the body part, simulating variation in the 3D anatomical model, attributable to the implantation of the stent, for each of a plurality of simulation conditions including one or more of the type of stent or a stent implantation target location based on the 3D anatomical model, and displaying the simulation results for each of the plurality of simulation conditions on a screen using the variation in the 3D anatomical model.

At least one embodiment of the present invention has the advantage of displaying simulation results for a plurality of respective simulation conditions on a screen in conjunction with each other so that variations in a 3D anatomical model can be contrasted with each other.

At least one embodiment of the present invention has the advantage of displaying the numerical value of an evaluation criterion on a 3D anatomical model, displayed as simulation results for each of a plurality of simulation conditions, at a reference location.

At least one embodiment of the present invention has the advantage of aiding in the selection of the most appropriate stent that is to be implanted in a subject.

The present invention was derived from research conducted as a part of the Industrial Convergence Fundamental Technology Development Project sponsored by the Korean Ministry of Trade, Industry and Energy and the Korea Institute of Industrial Technology Evaluation and Planning [Project Management Number: 10044910; Project Name: Development of Integrated Software System for Supporting Diagnosis and Treatment of Cardiovascular Diseases based on 3D High-precision Simulation using Multiple Medical Images].

As described above, although the present invention has been described in conjunction with specific details, such as specific components and limited embodiments and drawings, these are provided merely to help the overall understanding of the present invention. The present invention is not limited to these embodiments, and various modifications and variations can be made based on the foregoing description by those having ordinary knowledge in the art to which the present invention pertains.

Therefore, the technical spirit of the present invention should not be defined based on only the described embodiments, and the following claims, all equivalents to the claims and equivalent modifications should be construed as falling within the scope of the spirit of the present invention.

What is claimed is:

1. A medical image display method, comprising:
acquiring, by a processor, an image of a body part of a subject where a stent is to be implanted and a three-dimensional (3D) anatomical model which is generated to include the body part;
simulating, by the processor, variation in the 3D anatomical model, attributable to implantation of the stent, for each of a plurality of simulation conditions, including one or more of a type of stent and a target location where the stent is to be implanted, based on the 3D anatomical model; and
displaying, by the processor, simulation results for the plurality of respective simulation conditions on a screen by using variations in the 3D anatomical model.

2. The medical image display method of claim 1, wherein the displaying comprises displaying the simulation results for the plurality of respective simulation conditions on the screen in conjunction with each other so that the variations in the 3D anatomical model are contrasted with each other.

3. The medical image display method of claim 1, wherein the displaying comprises displaying the simulation results for the plurality of respective simulation conditions on the screen based on a same reference point within the 3D anatomical model.

4. The medical image display method of claim 1, wherein the displaying comprises displaying the simulation results for the plurality of respective simulation conditions on the screen based on a reference point or a reference direction at or in which the variations in the 3D anatomical model are maximized.

5. The medical image display method of claim 1, wherein the displaying comprises displaying one or more evaluation criteria, selected according to a predetermined setting or input of a user, on the 3D anatomical model based on the simulation results for the plurality of respective simulation conditions.

6. The medical image display method of claim 1, further comprising providing, by the processor, a user menu that enables a user to select one or more evaluation criteria to be displayed on the screen for the simulation results;
wherein the displaying comprises displaying the simulation results on the screen based on the evaluation criteria selected according to a response of the user via the user menu.

7. The medical image display method of claim 1, further comprising:
providing, by the processor, a user menu that enables a user to select a reference location at which numerical values of one or more evaluation criteria are to be displayed; and
identifying, by the processor, the reference location from the input of the user via the user menu;
wherein the displaying comprises displaying the numerical values of the evaluation criteria on the 3D anatomical model, displayed as the simulation results for each of the plurality of simulation conditions, at the reference location.

8. The medical image display method of claim 5, wherein the displaying comprises displaying the evaluation criteria, including one or more of a fractional flow reserve (FFR) value, a wall stress shear (WSS) value, a velocity value and a wall stress value, on the 3D anatomical model.

9. The medical image display method of claim 1, further comprising:
identifying, by the processor, an input of a user corresponding to any one of a plurality of images displayed on the display screen as the simulation results for the plurality of respective simulation conditions; and
storing information, including the image corresponding to the input of the user, in a database in response to the identified input of the user.

10. A medical image display system, comprising a processor configured to:
acquire an image of a body part of a subject where a stent is to be implanted and a three-dimensional (3D) anatomical model which is generated to include the body part;
simulate variation in the 3D anatomical model, attributable to implantation of the stent, for each of a plurality of simulation conditions, including one or more of a type of stent and a target location where the stent is to be implanted, based on the 3D anatomical model; and
display simulation results for the plurality of respective simulation conditions on a screen by using variations in the 3D anatomical model.

11. The medical image display system of claim 10, wherein the processor is further configured to display the simulation results for the plurality of respective simulation conditions on the screen in conjunction with each other so that the variations in the 3D anatomical model are contrasted with each other.

12. The medical image display system of claim 10, wherein the processor is further configured to display the simulation results for the plurality of respective simulation conditions on the screen based on a same reference point within the 3D anatomical model.

13. The medical image display system of claim 10, wherein the processor further configured to display the simulation results for the plurality of respective simulation conditions on the screen based on a reference point or a reference direction at or in which the variations in the 3D anatomical model are maximized.

14. The medical image display system of claim 10, wherein the processor is further configured to display one or more evaluation criteria, selected according to a predetermined setting or input of a user, on the 3D anatomical model based on the simulation results for the plurality of respective simulation conditions.

15. The medical image display system of claim 10, wherein the processor is further configured to:
provide a user menu that enables a user to select one or more evaluation criteria to be displayed on the screen for the simulation results; and
display the simulation results on the screen based on the evaluation criteria selected according to a response of the user via the user menu.

16. The medical image display system of claim 10, wherein the processor is further configured to:
provide a user menu that enables a user to select a reference location at which numerical values of one or more evaluation criteria are to be displayed;
identify the reference location from the input of the user via the user menu; and
display the numerical values of the evaluation criteria on the 3D anatomical model, displayed as the simulation results for each of the plurality of simulation conditions, at the reference location.

17. The medical image display system of claim 14, wherein the processor is further configured to display the evaluation criteria, including one or more of a fractional flow reserve (FFR) value, a wall stress shear (WSS) value, a velocity value and a wall stress value, on the 3D anatomical model.

18. The medical image display system of claim 10, wherein the processor is further configured to identify an input of a user corresponding to any one of a plurality of images displayed on the display screen as the simulation results for the plurality of respective simulation conditions; and
further comprising:
a database configured to store information, including the image corresponding to the input of the user, in response to the identified input of the user.
